(19) United States
(12) Patent Application Publication
(10) Pub. No.: US 2016/0082285 A1
(43) Pub. Date: Mar. 24, 2016

(54) APPLICATOR APPARATUS FOR PERFORMING BRACHYTHERAPY AND/OR MAGNETIC RESONANCE IMAGING

(71) Applicant: SIEMENS AKTIENGESELLSCHAFT, Munich (DE)

(72) Inventors: David GRODZKI, Erlangen (DE); Annemarie HAUSOTTE, Erlangen (DE); Bjoern HEISMANN, Erlangen (DE); Arne HENGERER, Moehrendorf (DE); Mark Aleksi KELLER-REICHENBECHER, Sandhausen (DE); Sebastian SCHMIDT, Weisendorf (DE)

(73) Assignee: SIEMENS AKTIENGESELLSCHAFT, Munich (DE)

(21) Appl. No.: 14/858,137

(22) Filed: Sep. 18, 2015

(30) Foreign Application Priority Data
Sep. 18, 2014 (DE) 102014218795.8

(51) Int. Cl. A61N 5/10; G01R 33/34; A61B 5/055

(52) U.S. Cl. CPC A61N 5/1007; A61B 5/055; A61N 5/1039; G01R 33/34084; A61N 2005/1011

(57) ABSTRACT

An applicator apparatus for performing brachytherapy and/or magnetic resonance imaging has an application main body for receiving at least one radiation source, and at least one antenna element connected to the application main body as a reception unit for a magnetic resonance device. The applicator apparatus can also include a processing module for a preamplification and/or digitization, the processing module being radiation-resistant and embodied to be separably connected to the antenna element, and the antenna element includes at least one part of the application main body.

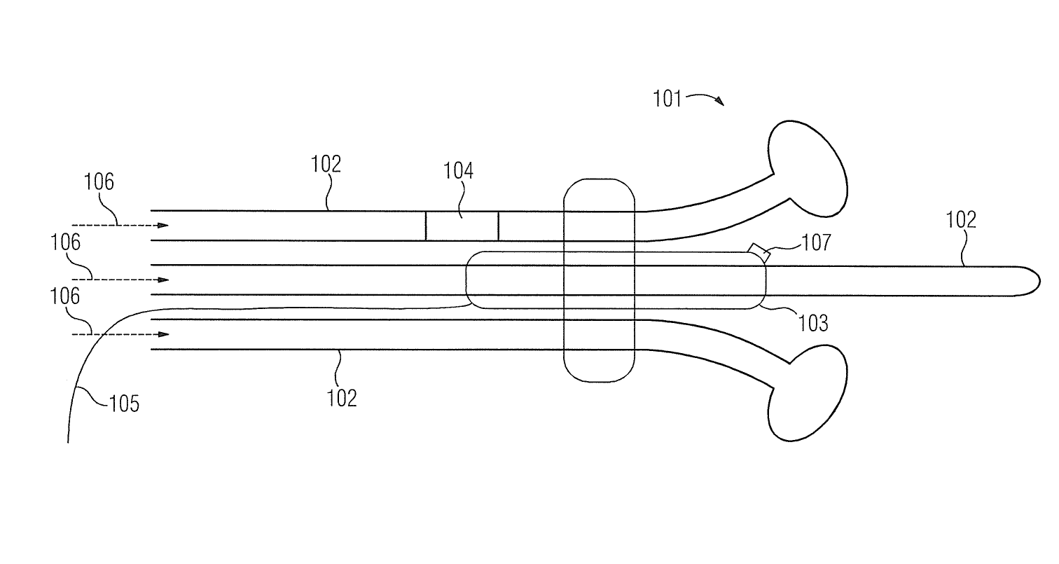

### BACKGROUND OF THE INVENTION

1. Field of the Invention

The present invention concerns an applicator apparatus for performing brachytherapy and/or magnetic resonance imaging, to a method for planning a brachytherapy and/or magnetic resonance imaging by means of an applicator apparatus and to a correspondingly embodied magnetic resonance device.

2. Description of the Prior Art

In brachytherapy, also known as internal radiation therapy, short distance radiation therapy, and therapy with enclosed radiation sources, radiation sources that include radioactive substances are introduced into the body of a patient, in order to damage or destroy target tissue, for instance tumors, using radiation locally in the body of the patient. In such cases the radiation exposure can be minimized for healthy body tissue, because the radiation sources can contain radioactive substances with a short radiation range, for instance low-energy Beta emitters or Gamma emitters. Moreover, the irradiation takes place locally from inside the body and need not first penetrate healthy tissue from outside of the body to reach the target tissue as occurs with an external radiation therapy using a linear accelerator. The radiation sources are typically introduced into the body by at least one applicator, often with a number of applicators.

Radiation sources implanted permanently in the body can be used for brachytherapy. These permanently implanted radiation sources are typically implanted in the vicinity of the target tissue by applicators embodied as capsules, also known as seeds. This method corresponds to brachytherapy by means of radiation sources, which have a low dose output, typically less than 2 Gray per hour. Such brachytherapy is also referred to as low-dose rate brachytherapy (LDR brachytherapy).

Alternatively, at least one applicator embodied as a hollow needle and/or catheter can be implanted in the body in the vicinity of the target tissue for the brachytherapy. By this means, temporary radiation sources, typically with a high dose output, typically of greater than 12 Gray per hour, can then be controlled. This method is also referred to as high-dose rate brachytherapy (HDR brachytherapy).

An irradiation plan is typically produced for a brachytherapy treatment, particularly if the at least one applicator is implanted into the body. This irradiation plan sets, for instance, how long and/or how often the radiation sources are to stay in the vicinity of the target tissue during an HDR brachytherapy. Alternatively or in addition, the irradiation plan can provide a recommendation for an implantation of further applicators, in particular with an LDR brachytherapy. The irradiation plan can naturally also provide further measures that appear useful to those skilled in this field.

In order to produce the irradiation plan, a dose calculation is typically required, particularly if the at least one applicator is already implanted in the body. For the dose calculation, knowledge that is as accurate as possible, relating to the position of the at least one applicator, is required, particularly with respect to the target tissue and/or surrounding tissue. Surrounding tissue may be, for instance, radiation-sensitive tissue at risk (organs at risk OAR) which is not provided for irradiation purposes.

In such cases the determination of the position of the applicator generally is implemented by computed tomography images when the radiation plan is being produced. A disadvantage of such images is that they have a minimal soft tissue contrast, particularly in regions in which tumors and radiation-sensitive tissue at risk are very close to one another, for instance the pelvis minor. Target organs such as e.g. the prostate or the cervix uteri are close to the organs at risk, such as the rectum and the neurovascular bundle, and can barely be distinguished because of the minimal soft tissue contrasts.

For this reason magnetic resonance images are increasingly used to produce such a radiation plan. One challenge is to achieve the best possible image quality during the shortest possible scan time. Particularly with therapies in the pelvis minor, external coils required therefor are situated relatively remotely from the target organ, which has an effect on the signal-to-noise ratio and thus on the image quality.

The use of intrarectal coils for producing the irradiation plan with magnetic resonance images is known. These coils are disposed close to the target organ and thus allow for an improved image quality. However, these coils are complicated to operate and the procedure has significant drawbacks in terms of patient comfort, since in addition to the brachytherapy applicators already introduced vaginally or transperineally, a rectal coil must also be introduced. Moreover, the coil can also cause changes to the anatomy, when it is removed for the actual treatment.

### SUMMARY OF THE INVENTION

An object of the invention is to provide an applicator apparatus for performing brachytherapy and/or magnetic resonance imaging, which achieves an improved image quantity and thus an improved and more accurate radiation plan.

This object is achieved in accordance with the invention by an applicator apparatus for performing a brachytherapy and/or magnetic resonance imaging that has an application main body for receiving at least one radiation source, and at least one antenna element connected to the application main body as a reception unit for a magnetic resonance apparatus.

The application main body is preferably embodied as a hollow needle and/or catheter and is configured to accommodate at least one radiation source. An antenna element, which is used as an integrated receive coil for a magnetic resonance apparatus, is connected thereto.

As a result, it is easily possible to reduce the distance between a target organ and the reception coil and thus to improve the image quality of a magnetic resonance image, and thereby to also improve the brachytherapy planning.

In a preferred embodiment, the applicator apparatus also includes a processing module for preamplification and/or digitization. In such cases a processing module is understood to mean an electronic circuit. The processing module can either be attached directly to the at least one antenna element or outside of an examination object to be examined with the applicator apparatus. Attachment outside of the examination object is advantageous since damage to the processing module by radiation is thus not expected.

In an embodiment, the processing module is attached to the at least one antenna element. A particularly compact embodiment of the applicator apparatus is achieved as a result.

In a further embodiment, the processing module is embodied to be radiation-resistant in the event that the electronics is attached close to the radiation source. Damage due to radiation is as a result prevented. This can be achieved for instance by semiconductor elements with larger structural widths than usual being used, which are then less radiation-sensitive. Alternatively, the processing module can also be shielded against radiation.

In a preferred embodiment, the processing module is configured to be separably connected to the antenna element. The processing module can thus be separated prior to an actual treatment taking place outside of a magnetic resonance system, in order to prevent damage due to the radiation.

In an embodiment, the antenna element includes at least one part of the application main body. This can be realized by tube-type elements of the application main body functioning as part of an antenna, for instance by being manufactured from a conductive material, or by conductor paths (runs) being attached to the tube-type elements of the application main body, which perform an antenna function. As a result, a particularly compact design of the applicator apparatus can be realized.

In a further embodiment, the application main body and/or the at least one antenna element are embodied as a single-use module. These allow for the rapid use of the application main body and/or antenna element, since any previously used parts do not have to be cleaned.

In a preferred embodiment, the connection between the application main body and/or the at least one antenna element is configured destructively. In such cases a destructive embodiment is understood to be a plug-in connection, which cannot be reused after separation, in order to prevent multiple usages.

In another embodiment, the processing module also includes an attenuation module with an attenuation map of the applicator apparatus. Since the application apparatus also attenuates the radiation, this must be taken into account during the planning. This is particularly important if the processing module is in the radiation path and this requires an additional shielding. A magnetic resonance image by itself is not readily suited to provide the attenuation data required therefor, but using the inventive procedure, provision of the geometry and attenuation of the applicator apparatus can be already provided in the device. In order to obtain such data, the applicator apparatus can be scanned in advance for instance with a computed tomography apparatus, or the data are generated from the construction data of the apparatus, since the attenuation is known for all used materials. This data then only need to be stored once in the attenuation module, and can then either be read out from the storage by the magnetic resonance apparatus or another interface that is suited for that purpose.

In a further embodiment, the attenuation module is configured for a deformation of the attenuation map. In the event that the applicator apparatus has a changeable, position-dependent geometry, attenuation data must exist for a number of positions of the applicator apparatus. In accordance with the invention, the attenuation module is able to determine a deformed attenuation data record, in other words the deformation of the attenuation map, from image data recorded from different positions of the applicator apparatus.

The present invention also encompasses a method for planning brachytherapy and/or magnetic resonance imaging using an applicator apparatus, and a magnetic resonance apparatus for planning brachytherapy and/or magnetic resonance imaging.

Further, the present invention encompasses a non-transitory, computer-readable data storage medium that can be loaded in a memory of a programmable controller or a computer of a magnetic resonance apparatus. All or various embodiments of the inventive method as described above can be implemented when programming instructions encoded in the storage medium are executed in the controller or control computer of the magnetic resonance apparatus. The programming instructions may require program means, e.g. libraries and auxiliary functions, in order to realize the corresponding embodiments of the method. The programming instructions may be a source code that must still be compiled and linked or that only has to be interpreted, or an executable software code, which for execution purposes only has to be loaded into the corresponding computing unit.

The computer-readable storage medium may be a DVD, a hard disk or a USB stick, on which electronically readable control information, in particular software, is stored.

The advantages of the inventive method, the inventive magnetic resonance apparatus and the inventive computer-readable storage medium essentially correspond to the advantages of the inventive applicator apparatus, which are explained above in detail.

### BRIEF DESCRIPTION OF THE DRAWINGS

The single FIGURE shows an inventive applicator apparatus.

### DESCRIPTION OF THE PREFERRED EMBODIMENTS

The FIGURE shows an inventive applicator apparatus 101. The applicator apparatus 101 includes an application main body 102, preferably embodied as a hollow needle and/or catheter, for receiving at least one radiation source 106 and an antenna element 103 connected to the application main body 102 as a receive unit for a magnetic resonance device.

It also includes a processing module 107 for preamplification and/or digitization purposes. The processing module 107 is attached here to the antenna element 103 and is configured in a radiation-resistant manner. This can be achieved for instance by semiconductor elements with larger structural widths than usual being used, which are then less radiation-sensitive. Alternatively, the processing module 107 can also be shielded against radiation.

The processing module 107 can alternatively be attached outside of an examination object to be examined with the applicator apparatus 101. Attachment outside of the examination object is advantageous since damage to the processing module 107 by radiation is thus not expected.

The antenna element 103 can however alternatively also include at least one part of the application main body 102. This can be realized by tube-type elements of the application main body 102 functioning as part of an antenna, for instance by being manufactured from a conductive material, or by conductor paths being attached to the tube-type elements of the application main body 102, which assume an antenna function.

The processing module 107 is separably connected to the antenna element 103. The processing module 107 can thus be separated prior to an actual treatment taking place outside of a magnetic resonance system in order to prevent damage due to the radiation.

The connection between the application main body 102 and/or the at least one antenna element 103 can alternatively also be embodied destructively. A destructive embodiment is preferably understood here to be a plug-in connection, which can no longer be reused after separation in order to prevent multiple usages.

The processing module 107 further includes an attenuation module 104 with an attenuation map of the applicator apparatus 101 and the attenuation module 104 is embodied for a deformation of the attenuation map. Since the applicator apparatus 101 also attenuates the radiation, this must be taken into account during planning. This is particularly important if the processing module 107 is in the radiation path and requires an additional shielding. Since a magnetic resonance image by itself is not readily suited to provide the attenuation data required therefor, it is possible to ensure by the inventive procedure provision of a geometry and attenuation of the applicator apparatus 101 already in the device. In order to obtain this data, the applicator apparatus 101 can be scanned in advance with a computed tomography device for instance, or the data are generated from the construction data of the apparatus, since the attenuation is known for all used materials. The data then only need to be stored once in the attenuation module 104 and can then either be read out from there by a magnetic resonance apparatus or another interface suited thereto. In the event that the applicator apparatus 101 has a changeable, position-dependent geometry, attenuation data must be available for a number of positions of the applicator apparatus 101. In accordance with the invention, the attenuation module 104 is able to determine a modified attenuation data record, in other words an appropriate modification of the attenuation map derived from image data sets respectively recorded from different positions of the applicator apparatus 101.

Although the invention has been illustrated and described in detail on the basis of the preferred exemplary embodiments, the invention is not limited by the disclosed examples and other variations can be derived therefrom by those skilled in the art without departing from the scope of the invention.

In summary, the invention provides an applicator apparatus for performing a brachytherapy and/or magnetic resonance imaging that includes an application main body for receiving at least one radiation source, and at least one antenna element connected to the application main body as a reception unit for a magnetic resonance apparatus.

In a preferred embodiment, the applicator apparatus also includes a processing module for a preamplification and/or digitization, the processing module is radiation-resistant and embodied to be separably connected to the antenna element and the antenna element includes at least one part of the application main body.

Although modifications and changes may be suggested by those skilled in the art, it is the intention of the inventors to embody within the patent warranted hereon all changes and modifications as reasonably and properly come within the scope of their contribution to the art.

We claim as our invention:

1. An applicator apparatus for selectively performing brachytherapy and magnetic resonance imaging, comprising:
an applicator body configured for introduction into a body cavity of a patient;
said applicator body comprising a radiation source that emits penetrating radiation into a patient via the body cavity; and
said applicator body comprising an antenna element configured to receive magnetic resonance signals within the body cavity of the patient.

2. An applicator apparatus as claimed in claim 1 comprising a processing module in communication with said antenna element, said processing module being configured for at least one of preamplification and digitization of said magnetic resonance signals.

3. An applicator apparatus as claimed in claim 2 wherein said processing module is attached to said antenna element in said applicator body.

4. An applicator apparatus as claimed in claim 3 wherein said processing module is comprised of radiation-resistant material.

5. An applicator apparatus as claimed in claim 2 wherein said processing module is situated externally of said applicator body.

6. An applicator apparatus as claimed in claim 1 wherein said antenna element comprises at least a portion of said applicator body.

7. An applicator apparatus as claimed in claim 1 wherein at least one of said applicator body and said antenna element is configured for one-time use.

8. An applicator apparatus as claimed in claim 7 wherein at least one of said applicator body and said antenna element is configured of disposable material.

9. An applicator apparatus as claimed in claim 1 comprising a processing module in communication with said antenna element, said processing module comprising an attenuation map of said applicator body that represents attenuation by said locator body of said penetrating radiation.

10. An applicator apparatus as claimed in claim 9 wherein said processing module is configured to modify said attenuation map dependent on respective positions of said applicator body in said body cavity.

11. A method for planning brachytherapy of a patient, comprising:
introducing an applicator apparatus into a body cavity of a patient, said applicator apparatus comprising a radiation source that emits penetrating radiation and a magnetic resonance reception coil;
within said body cavity, receiving magnetic resonance signals with said antenna element and, in a processor, reconstructing an image of a region of the patient from said magnetic resonance signals; and
in said processor, using said magnetic resonance image to generate a brachytherapy plan for operating said radiation source within said body cavity.

12. A method as claimed in claim 11 comprising storing, in said processor, an attenuation map that represents attenuation by said applicator body of said penetrating radiation, and generating said brachytherapy plan using said attenuation map.

13. A method as claimed in claim 12 comprising providing said processor with said attenuation map respectively for different positions of said applicator body, and generating said brachytherapy plan dependent on a selected position of said applicator body, and using the applicator map for that selected position.

14. A non-transitory, computer-readable data storage medium encoded with programming instructions, said storage medium being loaded into a processor that is in communication with a magnetic resonance reception coil that is mounted on an applicator apparatus that is introduced into a body cavity of a patient, said applicator apparatus also comprising a radiation source that emits penetrating radiation, and said programming instructions implementing brachytherapy planning by causing said processor to:
receive magnetic resonance signals from said antenna element and reconstruct an image of a region of the patient from said magnetic resonance signals; and
use said magnetic resonance image to generate a brachytherapy plan for operating said radiation source within said body cavity.

15. A storage medium as claimed in claim 14 wherein said programming instructions cause in said processor to access, from a memory, an attenuation map that represents attenuation by said applicator body of said penetrating radiation, and to generate said brachytherapy plan using said attenuation map.

16. A storage medium as claimed in claim 14 wherein said programming instructions cause said processor to access said attenuation map respectively for different positions of said applicator body, and to generate said brachytherapy plan dependent on a selected position of said applicator body, and to use the applicator map for that selected position.

17. A magnetic resonance apparatus comprising:
a magnetic resonance scanner;
an applicator body configured for introduction into a body cavity of a patient;
said applicator body comprising a radiation source that emits penetrating radiation into a patient via the body cavity;
said applicator body comprising an antenna element configured to receive magnetic resonance signals within the body cavity of the patient; and
a processor configured to use said magnetic resonance image to generate a brachytherapy plan for operating said radiation source within said body cavity.
